(19) United States
(12) Patent Application Publication
(10) Pub. No.: US 2025/0177151 A1
(43) Pub. Date: Jun. 5, 2025

(54) Customized Knee Replacement Implant Based on Average Geometry of the Knee

(71) Applicant: New York University, New York, NY (US)

(72) Inventors: Fatema Rahman, New York, NY (US); Hao Yang Chan, New York, NY (US); Gabriela Zapata, New York, NY (US); Peter Stanley Walker, New York, NY (US)

(73) Assignee: New York University, New York, NY (US)

(21) Appl. No.: 18/833,751

(22) PCT Filed: Jan. 30, 2023
(86) PCT No.: PCT/US2023/011877

§ 371 Date: Jul. 26, 2024

Related U.S. Application Data
(60) Provisional application No. 63/304,156, filed on Jan. 28, 2022.

(51) Int. Cl. A61F 2/38; A61F 2/30; A61F 2/46; B33Y 50/00; B33Y 80/00; G06F 30/27

(52) U.S. Cl. CPC A61F 2/389; B33Y 50/00; B33Y 80/00; G06F 30/27; A61F 2002/30952; A61F 2002/30985; A61F 2002/4658

(57) ABSTRACT

A total knee replacement implant having an anatomically representative femoral component that is based on an average knee geometry model determined from scans of multiple people. The implant can be customized for a given patient by scaling the average knee geometry according to one or more measurements (e.g. AP and ML) of the patient's knee. The implant can be formed by 3D printing or any suitable fabrication technique.

### CROSS-REFERENCES TO RELATED APPLICATIONS

This application claims the benefit of U.S. Provisional Application No. 63/304,156, filed on Jan. 28, 2022, and entitled “CUSTOMIZED KNEE REPLACEMENT IMPLANT BASED ON AVERAGE GEOMETRY OF THE KNEE”, the entirety of which is hereby incorporated by reference herein.

### BACKGROUND OF THE INVENTION

### 1. Field of the Invention

The present invention generally relates to knee implants having femoral components that are derived directly from anatomic shapes, including the femoral trochlea, the lateral bearing surface, the medial bearing surface, and the intercondylar area between them.

### 2. Description of the Related Art

The first condylar replacement type of total knee replacement intended for cruciate resection was the Freeman-Swanson (Freeman, Swanson, Todd 1977), designed in the late 1960's. This used a roller-in-trough geometry to provide stability and a large area of contact to minimize the wear. Also in the late 1960's, Gunston (1971) designed a conservative total knee consisting of independent runners embedded in the femoral and tibial condyles. In the early 1970's, Seedhom (1974) designed a total knee based on anatomical knee specimens, where he directly replicated the femoral surfaces and the tibial surfaces with the menisci present. A similar concept was described by Ewald in a patent. The Total Condylar knee was designed in the early 1970's with partially conforming bearing surfaces in the frontal and sagittal planes In order to provide an appropriate combination of laxity and stability (Walker, Wang, Masse 1974: Insall, Ranawat, Scott, Walker 1976). The relative radii were calculated to provide similar mechanical characteristics to that of the anatomic knee. The Kinematic Stabilizer and Insall-Burstein designs added an intercondylar cam-post mechanism to prevent anterior femoral subluxation and posterior femoral displacement in high flexion (Walker & Sathasivam, 2000; Robinson R D 2005). Since then, these ‘posterior stabilized’ (PS) designs have been modified and refined, and are now in widespread use. Typically the sagittal and frontal geometries are defined by connecting radii generally resembling the Total Condylar, while the intercondylar cam-post is designed separately, usually contacting from mid-flexion to maximum flexion. In such designs, the lateral and medial condyles are often symmetric, providing no lateral or medial bias to the motion. While symmetric designs can use simply derived geometry for both the femoral and tibial surfaces, as well as for cams and posts, such an approach is more difficult if anatomic motion patterns are required. For any type of design where both of the cruciates are resected, the problem of replicating the constraints provided by these ligaments remains a challenge to this day. In particular, providing AP stability and rotational laxity throughout flexion, while inducing femoral rollback to achieve high flexion angles without posterior impingement, seems difficult to achieve with the bearing surfaces alone, even with a cam-post mechanism.

A further drawback of standard designs is that, due to the limited number of sizes, they only approximate the anatomical geometry, such that the metallic and plastic components do not closely replicate the bone and cartilage shapes which are removed at surgery. This results in altered ligament length patterns and muscle lever arms, which can cause instability, tightness in certain parts of the flexion arc, or inefficient use of muscles, or even pain such as in the patello-femoral joint.

More recently, designs have been produced which provide greater medial than lateral constraint. One design concept, the medial pivot, uses a ball-in-a-socket for the medial compartment, and surfaces of low constraint on the lateral side (Blaha 2004; Moonot, 2009). Another design, the Journey Knee (Reis, Victor, Bellemans 2006, Victor Bellemans 2006), has a more constrained medial side and a cam-pivot which results in more posterior femoral displacement laterally. These designs are intended to achieve more normal kinematics, a goal that is receiving more attention today in an effort to improve function, especially in more active patients. Still more recently, designs have relied upon surfaces that were modified, flattened versions of average anatomy, as described in U.S. Pat. No. 9,907,662. So far however, these designs do not completely replicate anatomic motion and laxity-stability characteristics, or may have certain motion abnormalities in some patients. Hence there is still a need for a design that will allow close restoration of anatomic geometry and normal kinematics, and provides reliability and reproducibility.

### BRIEF SUMMARY OF THE INVENTION

In one aspect, the invention pertains to a customized knee implant that more closely matches the natural morphology of a patient's knee joint based on an average knee geometry and one or more measurements of the patient. In an exemplary embodiment, an average knee geometry model is determined from scans from multiple people or patients, one or more measurements are taken of the patient (e.g. two or more linear measurements, typically anterior-posterior (AP) and mediolateral width (ML) measurements of the patient's knee) and the average knee model is scaled linearly according to those measurements. In some embodiments, the knee model is also scaled in superior and inferior directions by an average scaling factor of the AP and ML scaling factors. In some embodiments, the scaled implant is an anatomically representative femoral component. In some embodiments, the implant further includes an anatomically representative femoral and tibial component, each based on the average knee design and scaled by the one or more measurements. In some embodiments, the custom implant is made by 3D printing or any suitable fabrication method.

In another aspect, the invention pertains to a method of making a customized knee implant with an anatomically representative femoral component that more closely matches the natural morphology of a patient's knee joint based on an average knee geometry and one or more measurements of the patient. Such methods can include: determining an average knee model from knee scans from multiple sample patients (e.g. 100 or more sample patients or any suitable number), obtaining one or more measurements of the patient (e.g. AP and ML measurements of the patient's knee), and scaling the average knee model linearly according to those measurements. In some embodiments, the knee model is also scaled in superior and inferior directions by an average scaling factor of the AP and ML scaling factors. Additionally, the method can include fabricating an anatomically representative tibial component scaled from the average knee geometry in the same manner. The overall tibial shape, particularly the peripheral shape, can be determined from the average tibial geometry. The method can include fabricating a femoral component by 3D printing or any suitable fabrication technique.

### BRIEF DESCRIPTION OF THE DRAWINGS

FIG. 1 is a distal-anterior view of a femoral component of a knee implant mounted on the distal femur, in accordance with some embodiments.

FIG. 2 is a medial-posterior view of the femoral component mounted on the distal femur, in accordance with some embodiments.

FIG. 3 is a distal-posterior view showing the femoral component (below) and the distal femur (above), in accordance with some embodiments.

FIG. 4 is a lateral-anterior view showing the femoral component (below) and the distal femur (above), in accordance with some embodiments.

FIG. 5 is a lateral view of the femoral component, in accordance with some embodiments.

FIGS. 6A-6D show different views of an average distal femur and average proximal tibia, and an average femoral component matching the anatomic femoral surfaces, in accordance with some embodiments.

FIG. 7 shows a set of femoral components of knee implants shown scaled to differing sizes, in accordance with some embodiments.

FIG. 8 graphically depicts the most prominent profiles of the lateral and medial condyles of the implant lined up posteriorly and distally, in accordance with some embodiments.

FIGS. 9A-9D show models of differences between the average knee model and the average of a first principal component, in accordance with some embodiments.

FIG. 10 shows a method of making a customized knee implant scaled from an average knee geometry based on measurements of the patient's knee, in accordance with some embodiments.

### DETAILED DESCRIPTION OF THE INVENTION

In one aspect, the invention generally relates to knee implants having femoral components that more closely match the natural morphology of the knee joint of a patient based on an average knee model and one or more measurements of the patient's knee.

Standard implants fail to replicate native knee kinematic functionality, due to mismatch of original condylar surfaces and non-anatomically placed implantation. (Daggett et al 2016; Saigo et al 2017). Thus, it is essential that the implant surface matches the original native knee to prevent instability and soft tissue impingement. One objective of current developments is to determine the ideal shapes and orientations of anatomically-shaped components, and test the accuracy of fit of the component surfaces.

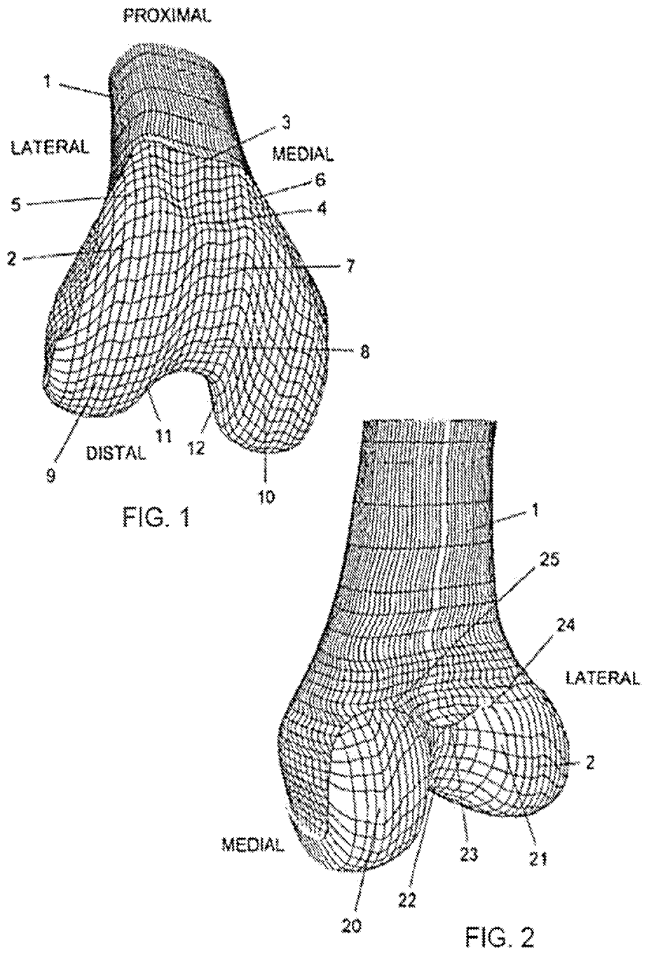

An exemplary knee replacement implant having an anatomically representative femoral component is shown in FIGS. 1-5. In FIGS. 1 and 2, the distal femur 1 is shown with the femoral component 2 fixed in place. The outer bearing surface of the femoral component is a replica of the anatomic distal femur for the purpose of illustration, but this can be modified slightly for purposes of smoothing, making surfaces of definable geometrical parameters, or manufacturability.

A baseline anatomically representative femur is defined as a femoral prosthesis comprising a distal bearing surface that duplicates that of an anatomic femur. The distal bearing surface shape can be taken from an individual natural femur. Alternatively, it can also be an average shape determined for a collection of cadaveric knees. It can also be an average shape determined from a collection of MRI scans, either normal knees, or knees with some degeneration as seen in osteoarthritis. The averaging method can either be by a scaling process followed by the definition of numerous sagittal slices and then averaging the slices, from which a new composite three-dimensional shape is made. It can also use a surfacing software which places a mean surface through a point cloud in space, the points determined directly from digitizing cadaveric knees, or determining points from MRI sections, where the initial step is to scale and position the point clouds.

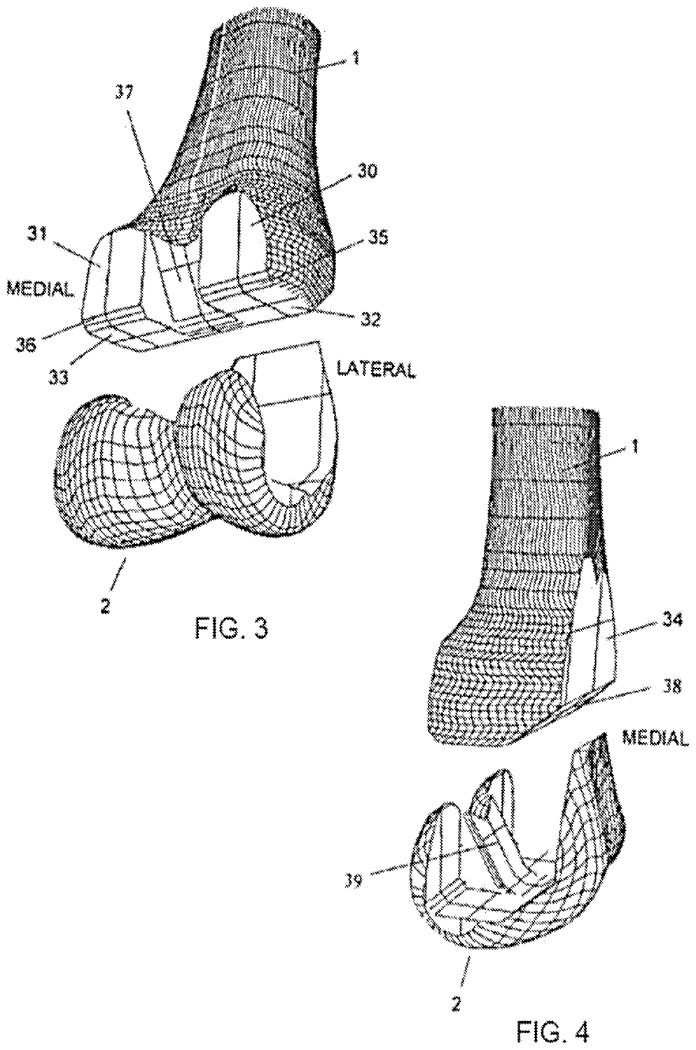

FIGS. 3 and 4 show the shape of the femoral component 2 and bone cuts which are required for fitting. The cuts shown are typical of standard faceted cuts used for fitting the femoral components of most of today's total knee systems. The facets consist of the postero-lateral 30, postero-medial 31, distal lateral 32, distal medial 33 and anterior 34. There are usually smaller additional facets at approximately 45 degrees to the above cuts 35, 36, 37, and 38. Of particular interest are the bone cuts used for the intercondylar area 39. These are shown as rectangular cuts which preserve sufficient wall thickness of the femoral component 2. The resulting shaped housing requires only a small amount of bone resection. In a sagittal view the anterior housing face makes an angle of approximately 60 degrees to the horizontal. In an alternative embodiment, the proximal surface of intercondylar area 39 may be formed as a smooth surface at least partially conformal with the distal surface of intercondylar area 39 thereby providing a constant wall thickness. The wall thickness in an exemplary embodiment is within the range of 2-4 mm. This configuration has the advantage of requiring the removal of less bone than the rectangular cut embodiment. In addition, the absence of rectangular corners minimizes the possibility of high stress areas. The required bone resection may be cut with a box chisel or curved rasp.

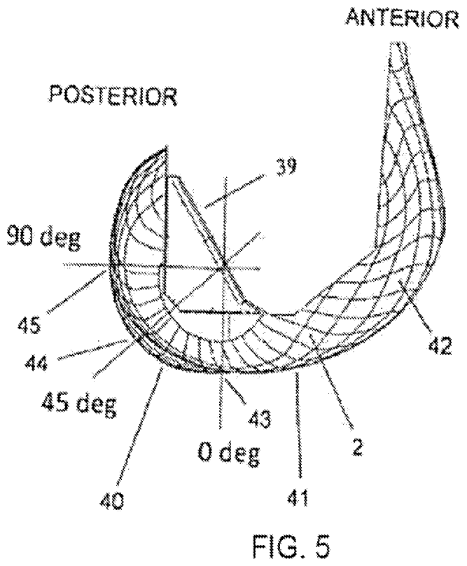

FIG. 5 shows a lateral view of the femoral component 2 with the positions of three sections. The section at 0 degrees flexion 43 is in a vertical frontal plane. The section at 45 degrees flexion 44 is halfway between a vertical frontal plane and a transverse horizontal plane. The section at 90 degrees flexion 45 is on a transverse horizontal plane. The sectional profiles of the lateral 41 and medial 40 condyles, differ and are based on the known anatomic shapes. This difference is important in terms of preserving the correct lengths of the lateral and medial collateral ligaments during the entire range of flexion-extension. The prominence of the patella flange on the lateral side 42 is a normal feature of the anatomic knee, which maintains the patella in the trochlea groove without lateral dislocation. The amount of prominence varies between patients, in particular between males and females. The prominence is generally less with females than males. In any case, the prominence can be reduced, in order to reduce the tensions in the anterior soft tissue structures, thereby facilitating a high flexion range.

In some embodiments, a baseline tibial surface may be generated from the mating femoral bearing surface as an envelope of the composite of multiple distal femoral bearing surfaces positioned, with respect to said tibial component, throughout the full range of flexure angles and axial rotation angles. The full range of flexion-extension typically extends from approximately 150 degrees flexion to −6 degrees extension while the full range of axial rotation angles is typically 10 to 20 degrees. The baseline tibial surface thus generated will exhibit complete conformity between the two surfaces in full extension, full flexion, and at the sides. While this will maximize contact area and minimize contact stresses, it is undesirable for three reasons. Firstly, it does not allow for any positional errors in placing the components at surgery. Secondly, any small manufacturing errors could result in contacts at the edges of the plastic tibial component. Thirdly in function, shear forces will cause the femoral component to contact the edges of the tibial surface, possibly resulting in deformity. Hence some lack of conformity between the femoral and tibial surfaces is desirable. For convenience this is preferably effected on the tibial surface. Starting with the generated baseline tibial surface, a proximal tibial bearing surface may be generated by modifying the baseline tibial surface, for example by increasing the sectional radii of the tibial surface so as cause a flattening out of the tibial surface.

In some embodiments, the replacement of the function of the cruciates and the menisci of the anatomic knee, may be obtained by interaction between the femoral and tibial bearing surfaces, so as to cause more normal medial pivot action, lateral femoral rollback in flexion, and roll-forward in extension. The embodiments herein presented incorporate features, in addition to those of the previous design, comprising retention of the intercondylar tibial eminences 78 and matching intercondylar femoral surfaces 22. These features provide the required medial-lateral constraint and also help to generate some of the anatomic motion characteristics described above. A further advantage of using anatomic surfaces, especially on the femur, is that anatomic patella tracking will occur, important for quadriceps mechanics. The addition of the Intercondylar Guiding Surfaces 22, provides a more definitive guidance to the pivotal motion. The Intercondylar Guiding Surfaces 22 cause the femur to displace posteriorly in flexion, but because of greater medial than lateral tibial dishing, most of the posterior displacement will occur on the lateral side, more closely resembling normal anatomic motion. The Intercondylar Guiding Surface 22 is designed to be in contact with tibial eminence 78 throughout flexion, providing a smooth motion and continuous guidance to the motion. The surfaces are rounded and always have contacts over discrete areas, rather than being small ‘point contacts’ at corners or edges. The Intercondylar Guiding Surfaces 22 may be configured to minimize the required bone resection. In some embodiments, normal medial pivot action may be enhanced by making the antero-medial femoral surface steeper. Femoral surface steepness may be increased by removal of material from the anterior portion of the medial condyle. This steeper medial condylar anterior surface when articulated with a correspondingly steeper anterior tibial surface, produces the desired anterior-posterior displacement stabilization.

### Average Knee Geometry

In an exemplary approach, the average knee geometry was obtained from a set of knee scans of a select patient population, namely patient with knees with intact functional knees. In some embodiments, the patient population includes a population of patients with early osteoarthritis, which is more representative of the knees being replaced, so as to obtain an accurate representative of the natural geometry of an intact functional knee joint. In some embodiments, this may be advantageous over customized implants developed based solely on a particular patient's knee morphology, particularly because a candidate for total knee replacement may have substantial deterioration of the femoral surface such that scans may no longer provide an anatomical representation of a healthy, intact knee joint.

In some embodiments, the select patient population includes any number of patients of any and all demographics. In this example, the patient population includes both female and male patients (e.g. about 60% female/40% male). It is appreciated that the patient population from which scans are obtained could be further refined (e.g. male patients, female patients, age range, height range, ethnicity, or any characteristic or demographic of the patient) so as to further refine an average knee model to better represent the patient. However, as the results discussed further below show, such refinements may not be needed to achieve highly accurate matches. Although it is appreciated that such refinements in determining the average knee model may provide additional improvements.

In one example, one hundred MRI scans of knees with early osteoarthritis were obtained from the NIH Osteoarthritis Initiative, converted into 3D meshes, and aligned via an anatomic coordinate system algorithm. Modeling software (e.g. Geomagic Design X) was used to determine the average anterior-posterior (AP) length. Each knee was then scaled to match the average AP length. A least squares algorithm was used to create the average surface model. This method was validated by generating a statistical shaped model using principal component analysis (PCA) to compare to the least square's method. While a particular methodology and software was used in this example, it is appreciated that an average knee model can be realized with any suitable software and by various other methodologies, including principal component analysis.

### Proof of Concept

To demonstrate the viability of using an average knee model, and in particular scaling the average model to fit a particular patient, a set of differing sizes of implants were developed to assess the fit with select knee joints. The average knee surface was used to design several different implant component systems. These implant systems include sizing schemes of 1 (single size), 3 sizes, 5 sizes, and 7 sizes, as set forth in Table 1. Custom components were created by scaling the AP and ML dimensions of the average component.

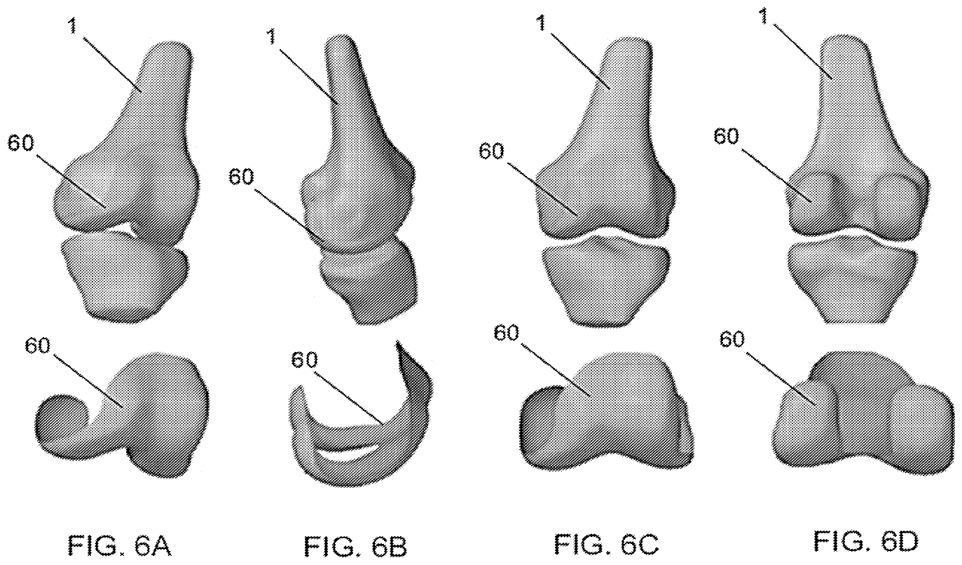

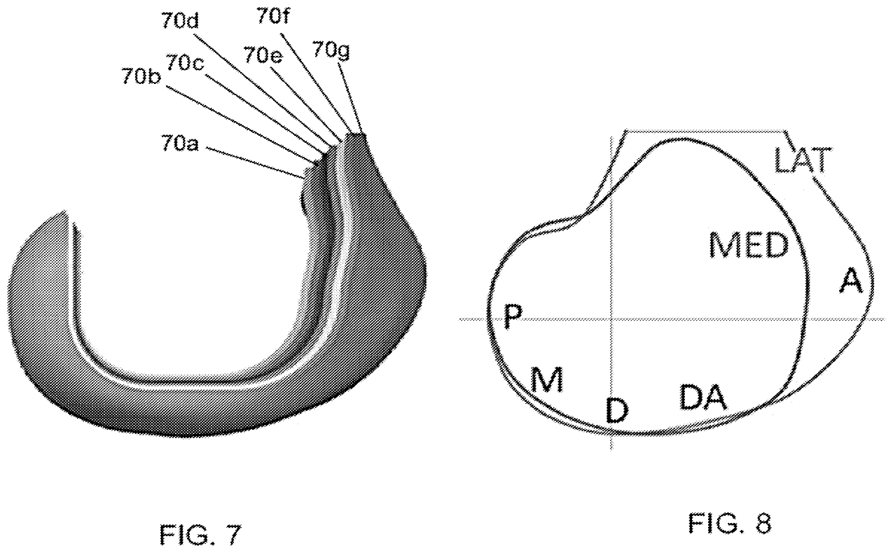

FIGS. 6A-D show various views of a femoral component 70 of a knee replacement implant based on average femoral design, as described herein. At top, each femoral component 60 is shown as attached to the femur 1, for example by the approach shown in FIG. 1. At bottom, each femoral component is shown separate from the femur to allow better visibility of the femoral component. FIG. 7 show exemplary femoral components of a knee replacement implant scaled to differing sizes. In this embodiment, the femoral components are scaled to 7 different sizes 70A-70G. FIG. 8 shows the show the most prominent profiles of the lateral and medial condyles, lined up posteriorly and distally.

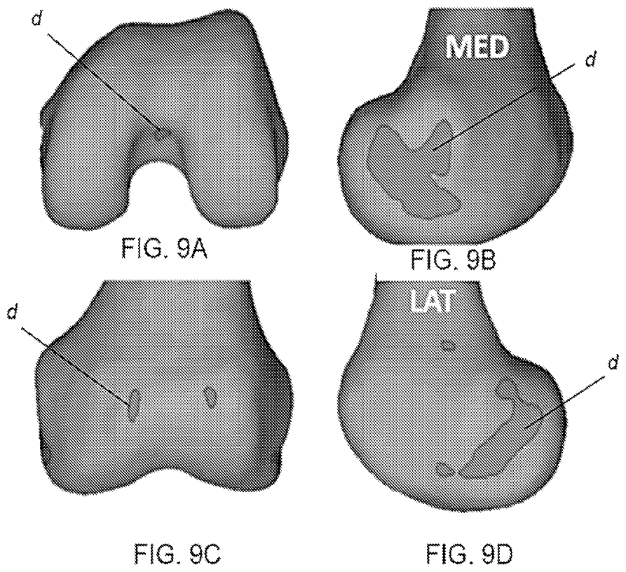

A further fifty arthritic knees were modelled to test accuracy of fit for all component sizing schemes. The closest fit implant components were lined up posteriorly and distally on each of the 50 femurs. Standard deviation maps were created to analyze error of fit of the implant surface compared to the native femur surface, as shown in FIGS. 9A-9C.

Average shape model derived from Principal Component Analysis had a discrepancy of 0.01 mm and a standard deviation of 0.05 mm when compared to the least squares fit. Statistical analysis of the mesh deviation maps between the femoral condylar surface and the components showed a decrease in deviation with larger number of sizes reducing deviations from 1.5 mm for a 1-size system, to 0.88 mm for a 7-size system, as shown in Table 1. The femoral components of a 5-size or 7-size system showed a best fit less than 1 mm. A fit within 1 mm or less represents an acceptable tolerance.

These results showed that a 5-size to 7-size system was sufficient, along with two widths for each size to avoid overhang. Accordingly, in some embodiments the set of scaled implants include 5 or more sizes, typically 7 or more sizes. Components based on the average anatomic shapes were an accurate fit on the bearing surfaces, but surgery to within 1-millimeter accuracy was needed. This study demonstrated that the approach of total knee design based on an average knee model provided more accurate reproduction of the normal knee bearing surfaces. Further, the results of the study showed that an accurate match of the femoral bearing surfaces was achieved to better than 1 millimeter if the component geometry was based on that of the average femur. Thus, this approach can be implemented in a customized knee implant based on the average knee model and measurements of the patient's knee to achieve even higher accuracy (e.g. within 1-millimeter, an average of about 0.75 mm, or an average of about 0.5 mm).

FIGS. 9A-9D show a comparison between the average femur and the average from the first principal component of the point cloud method. The lighter regions represent a difference of less than 1 mm, and the darker regions (d) indicate regions with differences greater than 1 mm. As can be seen, the main differences appear around the irregular epicondylar regions. In all situations, the main mismatch was on the superior patella flange, with maximum projection or undercut of 2 millimeters. The difference between select multiple sizes scaled from the average femur are shown in Table 1 below:

TABLE 1
Deviations of Differing Scaled Implant Sizes

| | MEAN OF | MEAN |
|---|---|---|
| NUMBER | AVERAGE | STANDARD |
| OF SIZES | DEVIATION | DEVIATION |
| IN SYSTEM | mm | mm |
| 1 | 1.21 | 1.02 |
| 3a | 0.99 | 0.85 |
| 3b | 0.99 | 0.86 |
| 3c | 1.02 | 0.86 |
| 5 | 0.93 | 0.80 |
| 7 | 0.88 | 0.74 |

As shown in Table 1, the first column shows the number of femoral component sizes in the system. The second column shows the mean of the deviations in millimeters between the femoral surface and the best-fit implant surface for the 50 femurs. The third column shows the mean standard deviation of the surface deviations for the 50 femurs. These differing sizes not only demonstrate the potential utility of a set of differing sizes from which an implant can be selected for a given patient, but further demonstrate the feasibility of scaling an average femoral component design based on measurements of a patient's femur (e.g. AP and ML) to provide highly accurate fit and superior performance as compared to conventional knee implants.

Thus, the study set forth above demonstrates that femoral implants based on an average knee geometry that are scaled to different sizes according to measurement of the patient's knee can provide an accurate femoral surface that matches a patient's knee morphology within an acceptable tolerance for improved fit and performance that more closely matches the natural geometry of the knee. Further, this approach can be further improved by customizing the implant to scale according to the measurements of the particular patient. The custom component surfaces matched to within an average of 0.5 mm. This customized approach can further reduce any mismatch, such as that seen on the superior patella flange.

### Custom-Fabrication

Accordingly, by utilizing the scaling approach based on average knee geometry described above, a total knee replacement implant can be custom-made for a particular patient based on the measured parameters of the patient's knee. By this approach the tolerance is even more precise than the multiple sizes noted above, and attain a tolerance even closer than 1 mm, typically matching surfaces within an average of 0.5 mm.

In one aspect, the implant utilizes a femoral component, which can be formed conventionally from a metal alloy, or formed of a polymer or resin material. In some embodiments, the femoral component can be formed by 3D printing. In some embodiments, the implant can further utilize an anatomically representative tibial component. The tibial component can similarly include an overall tibial shape, particularly the peripheral shape, that is designed based on the average knee design from scans of the multiple sample patients, and can similarly be scaled based on the one or more measurements (e.g. AP and ML) of the patient's knee. It is noted that the bearing surfaces of the tibial component can be generated from moving the femoral surface into multiple positions.

### Methods

In one aspect, the methods include determining an average knee geometry from a set of knee scans from multiple sample patients, scaling an implant design based on the average knee geometry according to a differing size based on one or more measurements (e.g. AP and ML) of a particular patient, and selecting and/or creating a custom implant based on the average knee geometry and the one or more measurements of the patient. As described previously, the implant can be selected from a set of differing sizes scaled from the average knee geometry. Preferably, the implant is custom-made such that the implant can be scaled precisely to the patient's knee measurement, thereby achieving closely matched tolerances and providing superior fit and implant that more closely matches a natural intact knee joint. The implant can be made by 3D printing or any suitable method.

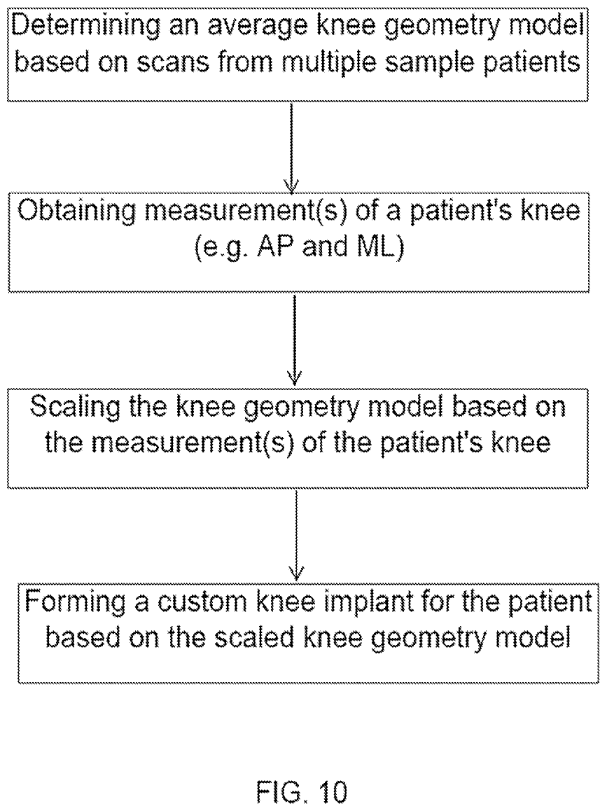

FIG. 10 depicts an exemplary method of making a total knee replacement implant based on an average knee mode. FIG. 10 shows a method of making a customized knee implant that includes steps of: determining an average knee geometry model based on scans from multiple people: obtaining measurements of the patient's knee (e.g. AP and ML); scaling the knee geometry model based on the measurements: and forming a custom knee implant for the patient based on the scaled knee geometry model.

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference herein for all purposes, including additional or alternative details, features and/or technical background.

What is claimed is:

1. A method of providing a knee replacement implant having at least a femoral component, the method comprising:
generating an average knee geometry of an anatomically representative femoral component based on a plurality of scans of a plurality of sample patients; and
obtaining one or more measurements of a given patient's knee:
scaling the average knee geometry according to the one or more measurements of the patient's knee: and
fabricating a custom implant based on the scaled knee geometry and the one or more measurements to achieve an accurate match for the patient's knee.

2. The method of claim 1 wherein the one or more measurements comprise an anterior-posterior (AP) and mediolateral width (ML) measurements.

3. The method of claim 2 wherein the average knee geometry is scaled linearly according to the AP and ML measurements.

4. The method of claim 3 wherein the method further comprises:
scaling the average knee geometry in the superior and inferior directions by an average scaling factor of the AP and ML scaling factors.

5. The method of claim 1 wherein the customized implant provides a match that is accurate to within 1 mm over all bearing surfaces of a femoral component of the implant.

6. The method of claim 1 wherein the plurality of sample patients comprise at least 50 people.

7. The method of claim 1 wherein the plurality of sample patients comprise at least 100 people.

8. The method of claim 1 wherein fabricating the implant comprises forming the implant by 3D printing.

9. The method of claim 2 further comprising:
generating an average knee geometry of an anatomically representative tibial component based on the plurality of scans of the plurality of sample patients.

10. The method of claim 9 wherein the method further comprises:
scaling the average knee geometry for the tibial component in the superior and inferior directions by an average scaling factor of the AP and ML scaling factors.

11. A knee replacement implant that is customized for a given patient, the implant comprising:
an anatomically representative femoral component:
wherein the femoral component has bearing surfaces that are based on an average knee geometry determined from a plurality of scans of a plurality of sample patients:
wherein the femoral component is scaled from the average knee geometry according to one or more measurements of the patient's knee.

12. The implant of claim 11 wherein the one or more measurements comprise an AP and ML measurement.

13. The implant of claim 12 wherein the average knee geometry is scaled linearly according to the AP and ML measurements.

14. The implant of claim 13 wherein the average knee geometry is further scaled in the superior and inferior directions by an average scaling factor of the AP and ML scaling factors.

15. The implant of claim 11 wherein the implant provides a match that is accurate to within 1 mm over all bearing surfaces of a femoral component of the implant.

16. The implant of claim 11 wherein the plurality of sample patients comprise at least 50 patients.

17. The implant of claim 11 wherein the implant is formed by 3D printing.

18. The implant of claim 12 wherein the implant further comprises:
an anatomically representative tibial component.

19. The implant of claim 18 wherein the tibial component has an overall geometry based on an average knee geometry determined from the plurality of scans of the plurality of sample patients.

20. The implant of claim 19 wherein the tibial component is scaled from the average knee geometry according to the AP and ML measurements of the patient knee.
